(19) United States
(12) Patent Application Publication
(10) Pub. No.: US 2026/0137436 A1
(43) Pub. Date: May 21, 2026

(54) ENHANCED SURGICAL TOOL SYSTEM

(71) Applicant: Christopher PEDICINI, Brentwood, TN (US)

(72) Inventor: Christopher PEDICINI, Brentwood, TN (US)

(21) Appl. No.: 19/397,494

(22) Filed: Nov. 21, 2025

Related U.S. Application Data
(60) Provisional application No. 63/723,282, filed on Nov. 21, 2024.

(51) Int. Cl. A61B 17/92; A61B 90/00

(52) U.S. Cl. CPC A61B 17/92; A61B 90/39; A61B 2090/3966

(57) ABSTRACT

A surgical tool may include a portion that interacts with an area associated with an environment and a set of fiducials disposed on the portion and arranged in a spatial configuration that is capable of defining a position, along at least two translational axes, and an orientation, along at least one rotational axis, of the portion relative to the area.

### CROSS-REFERENCE TO RELATED APPLICATION

This application claims the benefit of U.S. Provisional Application No. 63/723,282, filed Nov. 21, 2024, which is incorporated herein by reference in its entirety.

### BACKGROUND

Surgical tools are used to perform operations during surgical procedures. For example, a broach coupled to an impactor may be used to prepare a cavity in a bone of a patient to accommodate an implant.

### SUMMARY

In some aspects, the techniques described herein relate to a surgical tool, including: a portion that interacts with an area associated with an environment; and a set of fiducials disposed on the portion and arranged in a spatial configuration that is capable of defining a position and an orientation of the portion relative to the area, the position being along at least two translational axes, and the orientation being along at least one rotational axis.

In some aspects, the techniques described herein relate to a system, including: a surgical tool including a portion that interacts with an area associated with an environment; a set of fiducials disposed on the portion; a set of sensors that obtains data by detecting the set of fiducials; and circuitry configured to: determine, based on the data, a position and an orientation of the portion relative to the area, the position being along at least two translational axes, and the orientation being along at least one rotational axes.

### BRIEF DESCRIPTION OF THE DRAWINGS

FIGS. 1A-1E are diagrams of an example associated with an enhanced surgical tool system.

### DETAILED DESCRIPTION OF EXAMPLE EMBODIMENTS

The following detailed description of example implementations refers to the accompanying drawings. The same reference numbers in different drawings may identify the same or similar elements.

Surgical procedures require precise manipulation of surgical tools within a space (e.g., a three-dimensional anatomical space, among other examples). However, typical tracking systems cannot adequately determine a pose (e.g., a position and/or an orientation relative to an area within a surgical environment) of the surgical tools (e.g., during the surgical procedures). This inability leads to undetected misalignment which negatively affects the surgical procedures, such as by resulting in inaccurate operations (e.g., deviation from surgical plan) and increasing a risk of damage to surrounding structures (e.g., anatomical structures).

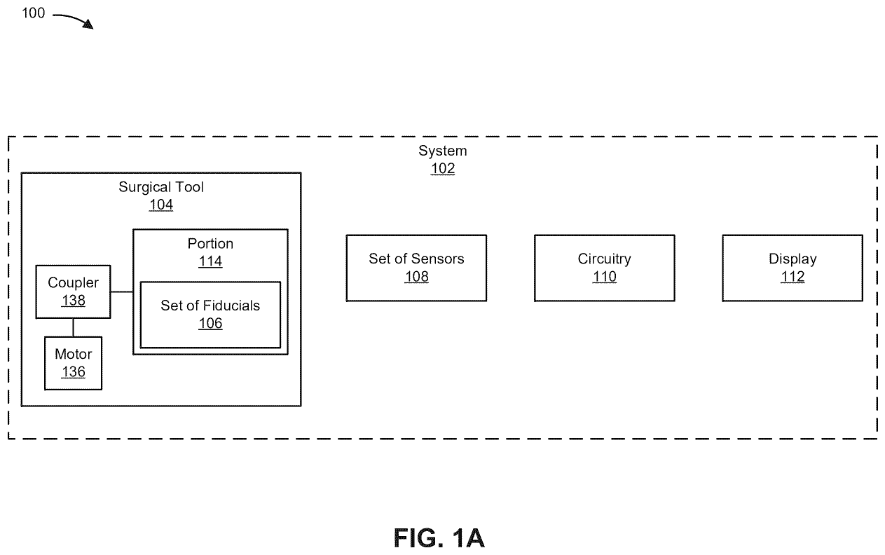

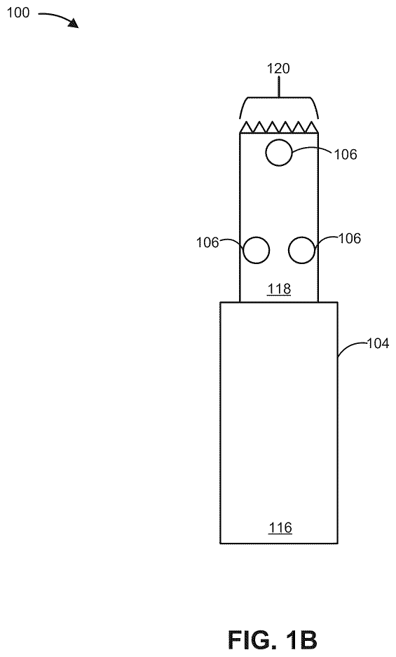

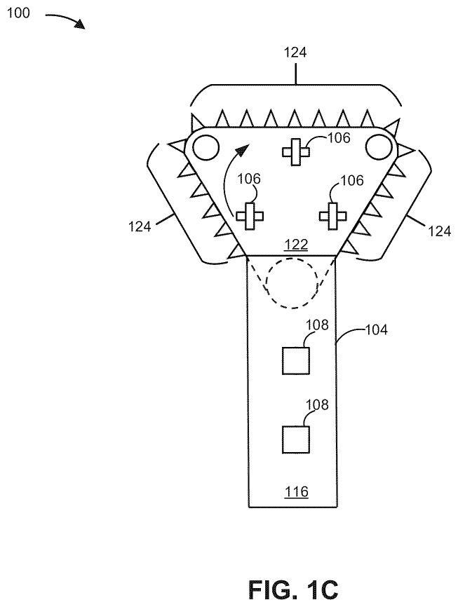

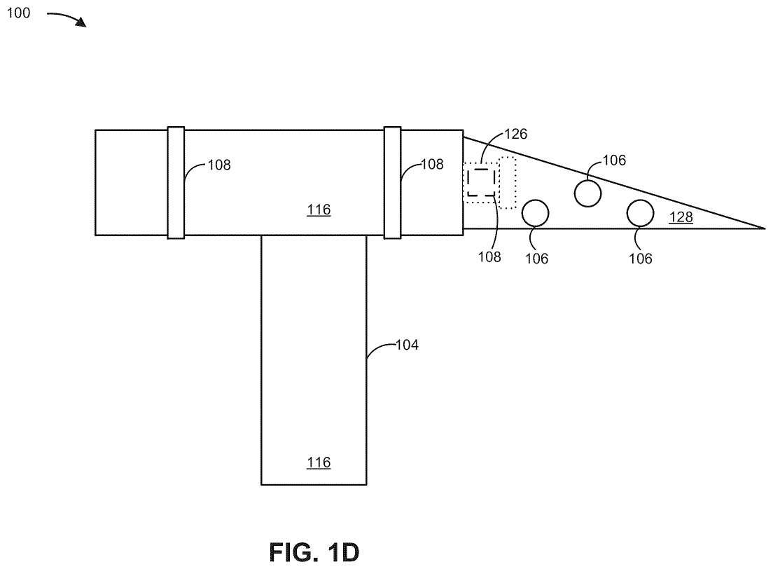

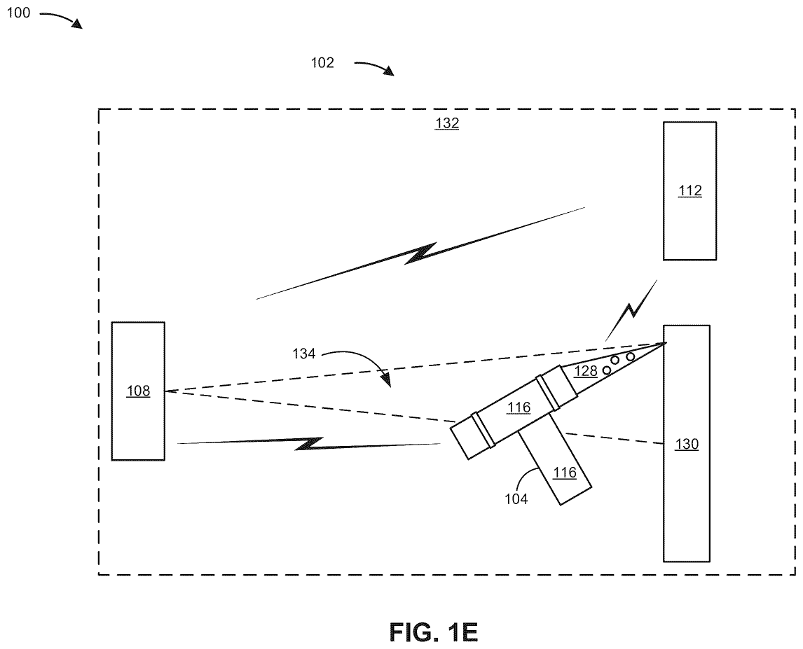

FIGS. 1A-1E are diagrams of an example 100 associated with a system 102 (e.g., an enhanced surgical tool system). As shown in FIG. 1A, the system 102 may include a surgical tool 104, a set of fiducials 106, a set of sensors 108, circuitry 110, and a display 112.

As shown in FIG. 1A, the surgical tool 104 may include a portion 114. For example, the portion 114 may be a body (e.g., shown as a body 116 in FIGS. 1B-1E), a cutting structure (e.g., shown as an oscillating saw blade 118 with cutting teeth 120 in FIG. 1B, and shown as a cutting assembly 122 with circulating cutting teeth 124 in FIG. 1C), an interface (e.g., shown as an adapter 126 in FIG. 1D), and/or a surgical implement (e.g., shown as a surgical implement 128 coupled to the surgical tool 104 in FIGS. 1D-1E), among other examples. Although the portion 114 is shown and described herein as being the body 116, the cutting structure, the interface, and/or the surgical implement, the portion 114 may be any suitable component associated with the system 102.

In some implementations, the set of fiducials 106 may be capable of being detected by the system 102. For example, the set of fiducials 106 may include one or more markers, features, and/or registration points, among other examples, capable of being detected by the set of sensors 108, among other examples.

In some implementations, the set of fiducials 106 may be disposed on one or more components associated with the system 102 to facilitate resolution of a position and/or an orientation of the one or more components associated with the system 102 relative to an area (e.g., shown as an area 130 in FIG. 1E) of an environment (e.g., shown as an environment 132 in FIG. 1E). For example, the set of fiducials 106 (e.g., disposed on the one or more components associated with the system 102) may include a number of fiducials, which may be based on a tracking resolution (e.g., a desired tracking resolution), arranged in a configuration (e.g., a deterministic spatial configuration that facilitates determination of a rigid body transformation, among other examples).

In some implementations, the set of fiducials 106 may include at least three fiducials arranged in an asymmetric, non-collinear configuration, and/or may include at least four fiducials arranged in a non-coplanar, volumetric configuration, which enables the system 102 to determine the position along three translational axes and/or the orientation along three rotational axes). Although the set of fiducials 106 is described as including at least three fiducials arranged in an asymmetric, non-collinear configuration, and/or as including at least four fiducials arranged in a non-coplanar, volumetric configuration, the set of fiducials 106 may include any suitable number of fiducials arranged in any suitable configuration (e.g., to enable the system 102 to resolve the position along any suitable number of translational axes and/or the orientation along any suitable number of rotational axes).

In some implementations, the set of sensors 108 may include one or more wired or wireless devices capable of receiving, generating, storing, transmitting, processing, detecting, and/or providing information associated with one or more components of the system 102 (e.g., the surgical tool 104, the portion 114, the area 130, and/or the environment 132, among other examples). For example, the set of sensors 108 may include a vision sensor (e.g., a machine vision sensor and/or a conformal vision sensor), an accelerometer, a gyroscope, a proximity sensor, a light sensor, a noise sensor, a pressure sensor, an ultrasonic sensor, a position sensor, a capacitive sensor, a timing device, an infrared sensor, an active sensor (e.g., a sensor that requires an external power signal), a passive sensor (e.g., a sensor that does not require an external power signal), a biological sensor, a magnetic sensor, an electromagnetic sensor, an analog sensor, and/or a digital sensor, among other examples.

The set of sensors 108 may sense and/or detect a condition and/or information and may transmit, using a wired or wireless communication interface, an indication of the detected condition and/or information, such as to one or more components associated with the system 102 (e.g., the surgical tool 104 and/or the circuitry 110, among other examples).

In some implementations, a procedure may be associated with information indicative of a trajectory (e.g., a reference trajectory or an intended trajectory indicated by a surgical plan and/or a template, among other examples) to be traversed by the surgical tool 104 (e.g., the portion 114, among other examples) to execute the procedure. For example, the intended trajectory may include a set of waypoints and/or a set of directional vectors that are aligned with the area 130 of the environment 132 (e.g., as indicated in the surgical plan and/or the template, among other examples).

In some implementations, the set of waypoints may be represented by a set of coordinates (e.g., a set of spatial coordinates, among other examples) associated with predetermined positions relative to the area 130 of the environment 132 and/or the set of directional vectors may be represented by a set of angles (e.g., a set of rotational angles, among other examples) associated with a set of predetermined orientations relative to the area 130 of the environment 132. Accordingly, for example, the set of waypoints may correspond to physical locations relative to the area 130 of the environment 132 and the set of directional vectors may correspond to physical angles relative to the area 130 of the environment 132. In this way, the surgical tool 104 (e.g., the portion 114, among other examples) may traverse, based on the set of waypoints and/or the set of directional vectors, the trajectory to execute the procedure (e.g., by executing the set of predetermined positions and/or the predetermined orientations during the procedure).

In some implementations, the set of fiducials 106 and/or the set of sensors 108 may be disposed on one or more components of the system 102. For example, the set of fiducials 106 (e.g., one or more markers, features, and/or registration points) may be disposed on the portion 114 (e.g., as shown in FIG. 1A), the oscillating saw blade 118 (e.g., as shown in FIG. 1), the cutting assembly 122 (e.g., as shown in FIG. 1C), and/or the surgical implement 128 (e.g., as shown in FIGS. 1D-1E), among other examples. In this way, the set of fiducials 106 may be tracked by the system 102, as described in more detail elsewhere herein.

As another example, the set of sensors 108 (e.g., one or more inertial measurement units (IMUs), accelerometers, gyroscopes, and/or conformal vision sensors) may be disposed on the body 116 (e.g., as shown in FIGS. 1C-1E), on the adapter 126 (e.g., as shown in FIG. 1D), and/or in the environment 132 (e.g., as shown in FIG. 1E), among other examples. In this way, the set of sensors 108 may obtain data associated with one or more components of the system 102, such as data associated with positions, orientations, linear velocities, angular velocities, references, and/or registration points associated with the system 102. Although the set of fiducials 106 and the set of sensors 108 are shown and described as being provided on one or more components of the system 102, the set of fiducials 106 and/or the set of sensors 108 may be disposed on any suitable component associated with the system 102.

In some implementations, the system 102 may track one or more components of the system 102 (e.g., e.g., via the circuitry 110 processing data obtained by the set of sensors 108, among other examples). For example, the system 102 may track the portion 114 as the portion 114 performs one or more operations during a procedure.

The set of sensors 108 may obtain data associated with the portion 114 during the procedure, such as by detecting the set of fiducials 106 disposed on the portion 114, by measuring parameters indicative of movement (e.g., when the set of sensors 108 is disposed on the portion and/or in proximity to the portion 114), and/or by detecting reference points and/or conditions associated with the area 130 of the environment 132, among other examples.

The set of sensors 108 may transmit, and the circuitry 110 may receive, the data associated with the portion 114. The circuitry 110 may process the data received from the set of sensors 108.

In some implementations, the circuitry 110 may determine, based on data associated with the portion 114, information indicative of the position of the portion 114, such as spatial coordinates (e.g., x, y, and/or z coordinates, among other examples) relative to the area 130 of the environment 132 and/or information indicative of the orientation of the portion 114, such as rotational angles (e.g., pitch, yaw, and/or roll angles, among other examples), relative to the area 130 of the environment 132. Accordingly, the circuitry 110 may resolve the position of the portion 114 along multiple translational axes and/or the orientation of the portion 114 along multiple rotational axes.

For example, the circuitry 110 may resolve the position of the portion 114 along at least two translational axes and the orientation of the portion 114 along at least one rotational axis. As another example, the circuitry 110 may resolve the position of the portion 114 along three translational axes and the orientation of the portion 114 along three rotational axes, among other examples. In this way, the circuitry 110 may resolve a six-degree of freedom pose of the portion 114 at one or more times during the procedure.

In some implementations, circuitry 110 may determine, based on the data associated with the portion 114, multiple positions and/or multiple orientations of the portion 114 relative to the area 130 of the environment 132 over time. For example, each position, of the multiple positions, and each orientation, of the multiple orientations, may represent a state (e.g., a pose) of the portion 114 at a corresponding time during the procedure.

In some implementations, the circuitry 110 may determine changes between the multiple positions of the portion 114 and/or changes between the multiple orientations of the portion 114 (e.g., over time intervals of the procedure). For example, the changes between the multiple positions may be indicative of a difference in spatial coordinates (e.g., x, y and, z coordinates) between two or more positions (e.g., determined at two or more times during the procedure). As another example, the changes between the multiple orientations may be indicative of a difference in rotational angles between two or more orientations (e.g., determined at two or more times during the procedure).

In some implementations, the circuitry 110 may determine one or more linear velocities (e.g., indicative of one or more rates of change of spatial coordinates over one or more time intervals of the procedure) of the portion 114 based on the changes between the multiple positions and/or may determine one or more angular velocities (e.g., indicative of one or more rates of change of rotational angles over one or more time intervals of the procedure) of the portion 114 based on the changes between the multiple orientations.

In some implementations, the circuitry 110 may predict one or more future positions and/or one or more future orientations of the portion 114 (e.g., corresponding to future times of the procedure), such as by extrapolating one or more linear velocities and/or one or more angular velocities over one or more future time intervals of the procedure to predict the one or more future positions and/or the one or more future orientations of the portion 114, among other examples.

In some implementations, the circuitry 110 may determine thresholds of translational movement and/or rotational movement of the portion 114 during the procedure (e.g., based on the trajectory of the procedure). For example, the circuitry 110 may determine a threshold of translational movement of the portion 114 (e.g., along the at least two translational axes) based on a threshold deviation of translational movement of the portion 114 from the set of waypoints of the trajectory. As another example, the circuitry 110 may determine a threshold of rotational movement of the portion 114 (e.g., along the at one rotational axis) based on a threshold deviation of rotational movement of the portion 114 from the set of directional vectors of the trajectory of the procedure.

In some implementations, the thresholds of translational movement and/or the thresholds of rotational movement may define an acceptable field of operation (e.g., shown as an acceptable field of operation 134 in FIG. 1E) associated with the surgical tool 104. For example, the acceptable field of operation may refer to a volumetric zone and/or an angular envelope relative to the area 130 of the environment 132 within which it is acceptable for the surgical tool 104 to operate (e.g., the acceptable field of operation may encompass the set of waypoints and/or the set of directional vectors of the intended trajectory while allowing for deviations within spatial and/or rotational movement tolerances, among other examples).

In some implementations, the circuitry 110 may determine whether the portion 114 is aligned with the area 130 of the environment 132 (e.g., at one or more times during the procedure) based on the thresholds of translational movement and/or the thresholds of rotational movement of the portion 114 relative to the area 130 of the environment 132. For example, the circuitry 110 may compare a position and/or an orientation of the portion 114 relative to the area 130 of the environment 132 (e.g., a current position and/or a current orientation of the portion 114 relative to the area 130 of the environment 132) and a reference position and/or a reference orientation of the portion 114 (e.g., indicated by the trajectory of the procedure) to determine whether the portion 114 is aligned with the area 130 of the environment 132.

For example, the circuitry 110 may determine that the position of the portion 114 relative to the area 130 of the environment 132 is misaligned based on a deviation of the position from a corresponding reference position satisfying the threshold of translational movement, such as by the position of the portion 114 exceeding a spatial tolerance of greater than or equal to 1 millimeter, among other examples. As another example, the circuitry 110 may determine that the orientation of the portion 114 relative to the area 130 of the environment 132 is misaligned based on a deviation of the orientation form a corresponding reference orientation satisfying the threshold of rotational movement, such as by the orientation of the portion 114 exceeding a rotational tolerance of greater than or equal to 2 degrees, among other examples.

In some implementations, the system 102 may include a motor (e.g., shown as a motor 136 in FIG. 1A) that drives one or more components associated with the surgical tool 104. For example, the motor 136 may drive the portion 114 and the portion 114 may provide impacts to the area 130 of the environment 132 in response to being driven by the motor 136. In some implementations, an operational speed of the motor 136 may increase from an initial operating speed in a controlled manner over time (e.g., according to a ramp profile, among other examples, to ensure that cuts or incisions are smoothly initiated). This prevents movements or vibrations that could compromise precision and reduces an initial torque exerted on the area 130 of the environment 132. This enhances control for an operator of the surgical tool 104, improves accuracy, minimizes a risk of accidental tissue damage during initial engagement, and reduces wear and tear on the surgical tool 104 by mitigating mechanical stress during start-up.

In some implementations, the system 102 may include a coupler (e.g., shown as a coupler 138 in FIG. 1A) that selectively couples the motor 136 to the surgical tool (e.g., to the portion 114 to drive the portion 114). The coupler 138 may decouple the motor 136 from the surgical tool 104 (e.g., from the portion 114) based on a force transmitted to the surgical tool 104 (e.g., the portion 114) satisfying a threshold.

In some implementations, the circuitry 110 may perform an action based on determining that the position of the portion 114 satisfies the translational movement threshold and/or based on determining that the orientation of the portion 114 satisfies the rotational movement threshold. For example, the circuitry 110 may modify (e.g., reduce and/or otherwise inhibit) an operational speed of the motor based on determining that the position of the portion 114 relative to the area 130 of the environment 132 satisfies the translational movement threshold and/or based on determining that the orientation of the portion 114 satisfies the rotational movement threshold.

In some implementations, the circuitry 110 may perform an action based on determining that a future position of the portion 114 satisfies a future translational movement threshold and/or based on determining that a future orientation of the portion 114 satisfies a future rotational movement threshold. For example, the circuitry 110 may modify (e.g., reduce and/or otherwise inhibit) an operational speed of the motor based on determining that the future position of the portion 114 relative to the area 130 of the environment 132 satisfies the future translational movement threshold and/or based on determining that the future orientation of the portion 114 satisfies the future rotational movement threshold.

In some implementations, the set of fiducials 106 may define a structural deflection, such as when the set of fiducials 106 is disposed on a cutting structure, such as being disposed on different portions of the oscillating saw blade 118. The circuitry 110 may determine, based on the data associated with the set of fiducials 106, a deflection value indicative of deflection occurring during the procedure, among other examples. For example, the circuitry 110 may detect differences caused by bending by comparing a first subset of fiducials at a first location of the cutting structure and a second subset of fiducials at a second location that is different from the first location against reference geometries, among other examples. In some implementations, the circuitry 110 may perform an action based on the deflection value satisfying a deflection threshold, such as modifying an operational speed of the motor.

In some implementations, the circuitry 110 may cause the display 112 to present a graphical rendering of the acceptable field of operation 134 (e.g., depicted as a virtual boundary and/or trajectory corridor overlaid the area 130 of the environment 132) and/or a real-time, or near real-time, representation of the position and/or the orientation of the surgical tool 104 (e.g., the portion 114) relative to the acceptable field of operation 134. The display may visually indicate when the surgical tool 104 (e.g., the portion 114) is operating within the acceptable field of operation 134 and/or when the surgical tool 104 (e.g., the portion) is not operating within the acceptable field of operation 134.

As indicated above, FIGS. 1A-1E are provided as an example. Other examples may differ from what is described with regard to FIGS. 1A-1E. The number and arrangement of the various components shown in FIGS. 1A-1E are provided as examples. In practice, there may be additional components, fewer components, different components, or differently arranged components than those shown in FIGS. 1A-1E. Additionally, or alternatively, a set of components (e.g., one or more components) shown in FIGS. 1A-1E may perform one or more functions described as being performed by another set of components shown in FIGS. 1A-1E.

Additionally, the functionality of the elements described herein may be implemented using circuitry or processing circuitry, including general-purpose processors, special-purpose processors, ICs, ASICs, conventional circuitry, or combinations thereof, configured or programmed to perform the disclosed functionality. A processor is a type of processing circuitry, as it includes transistors and other physical circuit components. A processor may execute instructions stored in a memory, thereby operating as a programmed processor. In this disclosure, the terms “circuitry,” “units,” or “means” refer to hardware that performs, or is programmed to perform, the described functionality. Such hardware may include any disclosed hardware or other known hardware that is configured or programmed to execute the described functions. When the hardware includes a processor, which is a type of circuitry, the circuitry, means, or units refer to a combination of hardware and software, where the software configures the hardware and/or processor to perform the specified functions.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

In the preceding specification, various example embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A surgical tool, comprising:
a portion that interacts with an area associated with an environment; and
a set of fiducials disposed on the portion and arranged in a spatial configuration that is capable of defining a position and an orientation of the portion relative to the area, the position being along at least two translational axes, and the orientation being along at least one rotational axis.

2. The surgical tool of claim 1, wherein the position is along three translational axes.

3. The surgical tool of claim 1, wherein the orientation is along three rotational axes.

4. The surgical tool of claim 1, wherein the set of fiducials is capable of defining a threshold of translational movement of the portion along the at least two translational axes.

5. The surgical tool of claim 1, wherein the set of fiducials is capable of defining a threshold of rotational movement of the portion along the at least one rotational axis.

6. The surgical tool of claim 1, wherein the portion is a body of the surgical tool.

7. The surgical tool of claim 1, wherein the portion is a cutting structure of the surgical tool.

8. The surgical tool of claim 1, wherein the portion is an adapter of the surgical tool.

9. The surgical tool of claim 1, wherein the portion is a surgical implement.

10. The surgical tool of claim 1, wherein the portion is a cutting structure of the surgical tool and the set of fiducials is capable of defining a deflection of the cutting structure.

11. The surgical tool of claim 1, further comprising:
a motor that increases an operational speed from an initial operating speed in a controlled manner over time.

12. The surgical tool of claim 1, further comprising:
a motor that drives the portion; and
a coupler that selectively couples the motor to the portion, the coupler decoupling the motor from the portion based on a force transmitted to the portion satisfying a threshold.

13. The surgical tool of claim 1, further comprising:
a motor that drives the portion and reduces an operational speed based on at least one of:
the position deviating from a reference position, or
the orientation deviating from a reference orientation.

14. The surgical tool of claim 1, further comprising:
a sensor that obtains data by detecting the set of fiducials; and
circuitry configured to:
determine, based on the data, the position and the orientation relative to the area; and
perform an action based on at least one of:
the position deviating from a reference position, or
the orientation deviating from a reference orientation.

15. The surgical tool of claim 14, further comprising:
a motor that drives the portion,
wherein the action is modifying an operational speed of the motor.

16. A system, comprising:
a surgical tool including a portion that interacts with an area associated with an environment;
a set of fiducials disposed on the portion;
a set of sensors that obtains data by detecting the set of fiducials; and
circuitry configured to:
determine, based on the data, a position and an orientation of the portion relative to the area, the position being along at least two translational axes, and the orientation being along at least one rotational axes.

17. The system of claim 16, wherein the circuitry is further configured to perform an action based on at least one of:
the position deviating from a reference position, or
the orientation deviating from a reference orientation.

18. The system of claim 17, wherein the action is modifying an operational speed of a motor that drives the portion.

19. The system of claim 16, wherein the circuitry is further configured to:
determine, based on the data, multiple positions and multiple orientations of the portion relative to the area over time;
predict, based on at least one of changes between the multiple positions or changes between the multiple orientations, a future position and a future orientation of the portion relative to the area, the changes between the multiple positions being indicative of linear velocities and the changes between the multiple orientations being indicative of angular velocities; and
perform an action based on at least one of:
the future position of the portion deviating from a future reference position, or
the future orientation deviating from a future reference orientation.

20. The system of claim 16, wherein the portion is a cutting structure and the circuitry is further configured to:
determine, based on the data, a deflection value indicative of a structural deflection of the cutting structure; and
perform an action based on the deflection value satisfying a deflection threshold.
